(19) United States
(12) Patent Application Publication
(10) Pub. No.: US 2026/0001059 A1
(43) Pub. Date: Jan. 1, 2026

(54) LIGHT ALKANE CHROMIUM-BASED DEHYDROGENATION CATALYST, AND PREPARATION METHOD AND APPLICATION THEREOF

(71) Applicant: REZEL ENGINEERING CO., LTD, Shanghai (CN)

(72) Inventors: Chunxue ZHANG, Shanghai (CN); Runsheng ZHUO, Shanghai (CN); Qiushi SUN, Shanghai (CN); Bing LIU, Shanghai (CN); Yusen RAO, Shanghai (CN); Xingyue LAN, Shanghai (CN); Ruiling ZHAO, Shanghai (CN); Xinsheng LIU, Shanghai (CN)

(21) Appl. No.: 18/992,491

(22) PCT Filed: Jul. 6, 2023
(86) PCT No.: PCT/CN2023/106184

§ 371 Date: Jan. 8, 2025

(30) Foreign Application Priority Data
Jul. 8, 2022 (CN) 202210806633.6

(51) Int. Cl. B01J 23/26; B01J 35/40; B01J 35/61; B01J 35/70; B01J 37/02; B01J 37/12; C07C 5/333

(52) U.S. Cl. CPC B01J 23/26; B01J 35/40; B01J 35/613; B01J 35/615; B01J 35/70; B01J 37/0213; B01J 37/12; C07C 5/3332; C07C 2523/26

(57) ABSTRACT

The present invention discloses a light alkane chromium-based dehydrogenation catalyst, and a preparation method and application thereof, and belongs to the field of petrochemical technology. The dehydrogenation catalyst has a spinel structure and comprises the following components in mass fractions based on the total weight on a dry basis: 0.1-35% of chromium oxide, 0.1-5% of a first promoter, 0.1-10% of a second promoter, 0.1-5% of a third promoter, and the balance being a fluidized bed carrier. This dehydrogenation catalyst controls the existence state of the active centers in the catalyst by adjusting the electrical properties of the surface of the carrier, resulting in higher dehydrogenation activity and propylene selectivity of the catalyst; at the same time, it enhances the overall stability, strength, and attrition of the catalyst, alleviating the loss problem of the catalyst during use.

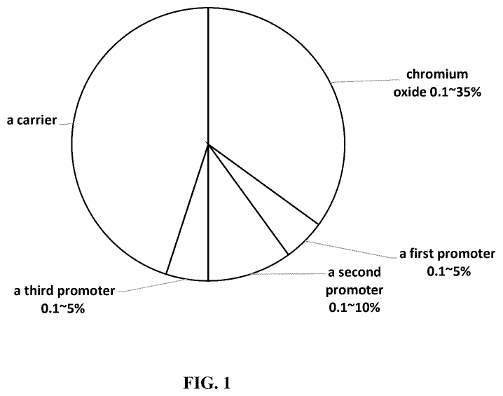

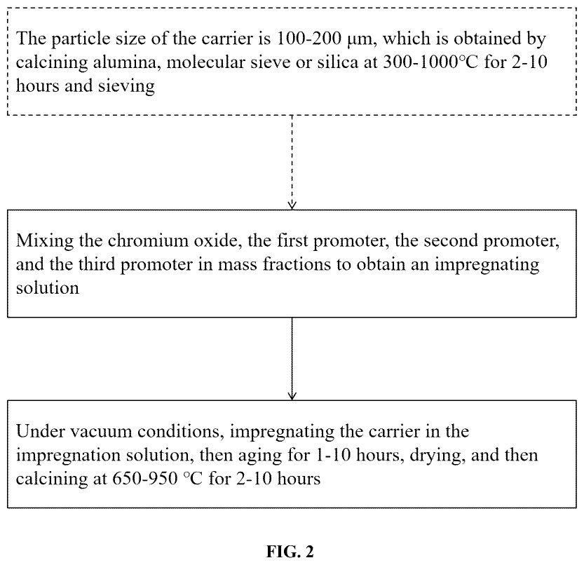

### TECHNICAL FIELD

The present invention belongs to the technical field of petrochemical technology, and specifically related to a light alkane chromium-based dehydrogenation catalyst, and a preparation method and application thereof.

### BACKGROUND

With the rapid growth of demand for derivatives such as polypropylene, polyacrylonitrile, propylene oxide, and acrylic acid, etc., the demand for propylene has also been increasing year by year. Currently, the growth rate of propylene demand in the global market has exceeded the growth rate of production capacity, and market supply and demand are becoming increasingly tight. In recent years, the demand for propylene in China has also grown rapidly. In processes such as catalytic cracking and steam cracking of naphtha, etc., a large amount of light alkanes are produced as by-products. Economically converting these alkanes into propylene will effectively improve the market demand for propylene. Compared with the methanol to olefin process and coal to olefin process, the dehydrogenation of alkane to olefin process has the advantages such as abundant raw materials, low cost, no need to introduce water in the reaction process, and low wastewater volume, etc. The light alkane dehydrogenation process can be divided into oxidative dehydrogenation and direct dehydrogenation, wherein the oxidative dehydrogenation reaction has high heat release, dangerous operation process, poor selectivity to target products, and low conversion rate, thus it has no advantages. Therefore, direct dehydrogenation of alkanes is the most effective method for producing olefins.

The currently developed direct dehydrogenation processes for alkanes include Oleflex process from UOP Company, Catofin process from AirProduct&Chemical Company, Phillips' Star process from Phillips Company, FBD process from Snamprogetti&Yarsintz Company, and Linde process from Linde Company. Based on the comprehensive comparisons of operating pressure, temperature, continuity, stable operation of production equipment, and reduction in production cost, the fluidized bed alkane dehydrogenation process has broad development prospects. Compared with fixed bed reactors and moving bed reactors, fluidized bed reactors have the advantages of high bed heat transfer efficiency, capability of stable control of uniform bed temperature, continuous reaction and regeneration, and long-term stable operation at higher temperatures, etc. The FBD process of Snamprogetti in Italy and Yarsintz in Russia adopts the fluidized bed reactors equipped with reaction and regeneration systems. Currently, it has been applied to one 130000 tons/year of isobutene industrial plant in Russia, as well as five isobutane and propane dehydrogenation plants. Meanwhile, China also attaches great importance to the development of fluidized bed alkane dehydrogenation process. The novel propane/butane dehydrogenation (ADHO) technology, independently researched and developed by the State Key Laboratory of Heavy Oil of China University of Petroleum (East China) and designed by the East China Design Branch of China Petroleum Engineering Construction Corporation, has been proved by industrial tests in Shandong Hengyuan Petrochemical Industry in 2016, filling the gap in this technology field in China.

In the direct dehydrogenation reaction of propane, the by-products are mainly C1 and C2 light hydrocarbons, which are generated from cracking. At present, most FBD processes adopt powdered Cr₂O₃ catalysts or microsphere Cr₂O₃/Al₂O₃ catalysts. Both the selectivity and stability of chromium-based catalysts need to be improved. Traditional chromium-based catalysts mostly use aluminum oxide as a carrier and are prepared by modifying with elements such as alkali metals, etc. Chinese patents CN110560043 and CN103769078, as well as US patent US20030232720 have mentioned the role of alkali metals in the dehydrogenation catalysts and disclose a method of adding a small amount of alkali metal elements to the catalyst in an attempt to reduce the acidity of the catalyst itself. However, due to the strong alkalinity of alkali metals, not only does it reduce acidity, but also it lowers the stability of the catalyst. The U.S. Pat. No. 8,835,347 adds some alkali metals and alkaline earth metals to the dehydrogenation catalyst in hopes of changing its selectivity. However, based on its preparation method and performance of the catalyst, it can be seen that the alkaline earth element added in this patent only acts to cover the acidic sites on the surface of the catalyst, which improves the selectivity. However, the alkaline earth element does not form a spinel structure with the carrier of the fixed bed, and thus the service life of its catalyst has not been enhanced.

Compared to fixed bed and moving bed reactors, fluidized bed reactors can stably control the uniform temperature of the bed layer, the heat transfer process is almost instantaneous, and have high heat and mass transfer efficiency. Therefore, the experimental study on catalytic dehydrogenation of propane in fluidized bed reactors has reduced the occurrence of side reactions caused by uneven temperature. Moreover, the fluidized bed dehydrogenation process can carry out continuous reaction and regeneration, achieving long-term stable operation at higher temperatures. However, fluidized bed reactors have high requirements for the fluidization performance and wear resistance of catalysts. At present, the propane conversion rate and propylene yield of catalysts used in fluidized bed processes are relatively low.

However, the existing catalysts for fluidized bed dehydrogenation processes often suffer from loss problem caused by abrasion of catalyst particles due to issues such as strength, abrasion, and stability, etc. On the one hand, it increases the consumption of the catalyst, and on the other hand, it also has high requirements for the cyclone separator and particle recovery device of the equipment. Therefore, it is necessary to improve the catalyst for fluidized bed dehydrogenation process to overcome the above problems.

### Contents of Invention

To overcome the problems of “insufficient catalyst strength, abrasion and stability in fluidized bed dehydrogenation process” in the existing technology, the present invention provides a light alkane chromium-based dehydrogenation catalyst, and a preparation method and application thereof.

The present invention is implemented by the following technical solutions:

In a first aspect, the present invention provides a light alkane chromium-based dehydrogenation catalyst, which has a spinel structure and comprises the following components in mass fractions based on the total weight on a dry basis:
0.1-35% of chromium oxide, 0.1-5% of a first promoter, 0.1-10% of a second promoter, 0.1-5% of a third promoter, and the balance being a fluidized bed carrier;
the first promoter is a substance containing at least one alkaline earth metal element;
the second promoter is a substance containing at least one Group IVB element;
the third promoter is a substance containing at least one lanthanide metal element.

Further, in a preferred embodiment of the present invention, the specific surface area of the above-mentioned fluidized bed carrier is 50-300 m²/g, and the particle size is 100-200 μm.

Further, in a preferred embodiment of the present invention, the above-mentioned fluidized bed carrier comprises alumina, molecular sieve, or silica with high mechanical strength.

The spinel structure includes at least one of a magnesia-alumina spinel structure, a calcium aluminum spinel structure, a titanium containing spinel structure and a perovskite structure.

Further, in a preferred embodiment of the present invention, the above mentioned first promoter comprises one or more of alkaline earth metal powders, alkaline earth metal halides, alkaline earth metal oxides, alkaline earth metal sulfides, alkaline earth metal nitrates, alkaline earth metal acetates and alkaline earth metal oxalates.

Further, in a preferred embodiment of the present invention, the above mentioned second promoter comprises one or more of metal powders of Group IVB elements, halides of Group IVB elements, oxides of Group IVB elements, sulfides of Group IVB elements, nitrates of Group IVB elements, acetates of Group IVB elements and oxalates of Group IVB elements.

Further, in a preferred embodiment of the present invention, the above mentioned third promoter comprises one or more of lanthanide metal powders, lanthanide metal halides, lanthanide metal oxides, lanthanide metal sulfides, lanthanide metal carbides, lanthanide metal nitrates, lanthanide metal acetates and lanthanide metal oxalates.

In a second aspect, the present invention provides a preparation method of a light alkane chromium-based dehydrogenation catalyst, comprising:
mixing chromium oxide, a first promoter, a second promoter and a third promoter in mass fractions to obtain an impregnating solution; and
under vacuum conditions, impregnating a fluidized bed carrier in the impregnation solution, then aging for 1-10 hours, drying, and then calcining at 650-950° C. for 2-10 hours.

Further, in a preferred embodiment of the present invention, the particle size of the above-mentioned fluidized bed carrier is 100-200 μm, which is obtained by calcining alumina, molecular sieve or silica at 300-1000° C. for 2-10 hours and sieving.

In a third aspect, the present invention provides application of the above-mentioned light alkane chromium-based dehydrogenation catalyst. The catalyst is used in a fluidized bed reactor with a reaction pressure of 0.01-0.50 MPa, a temperature of 530-660° C., and a volume space velocity of 800-2400 h⁻¹.

Compared with the prior art, the present invention at least has the following technical effects.

Compared to the existing traditional fluidized bed chromium-based dehydrogenation catalysts, the light alkane chromium-based dehydrogenation catalyst for fluidized beds provided by the present invention uses alkaline earth metals, Group IVB elements, and lanthanide metal elements. On the one hand, the existence state of the active centers in the catalyst is controlled by adjusting the electrical properties of the surface of the carrier, resulting in higher dehydrogenation activity and propylene selectivity of the catalyst; on the other hand, due to the formation of a relatively stable spinel structure between the promoter and fluidized bed carrier, the overall stability, strength, and abrasion of the catalyst are enhanced, alleviating the loss problem of the catalyst during use. In addition, the catalyst has simple and readily available raw materials, a simple preparation process, and can be efficiently, stably and economically produced using the existing production lines, effectively replacing the existing traditional chromium-based dehydrogenation catalysts.

### DETAILED DESCRIPTION OF THE EMBODIMENTS

The implementation methods of the present invention will be described in detail below in conjunction with examples. However, those skilled in the art will understand that the following examples are only used to illustrate the present invention and should not be regarded as limiting the scope of the present invention. The specific conditions not specified in the examples are carried out according to conventional conditions or conditions recommended by the manufacturer. The reagents or instruments used without indicating the manufacturer are all conventional products that can be available commercially.

The technical solution of the present invention is as follows:
to meet the high requirements of fluidized bed reactors for the fluidity and wear resistance of catalysts, and to solve the problems of poor stability and low selectivity of light alkane chromium-based dehydrogenation catalysts, the present invention provides a light alkane chromium-based dehydrogenation catalyst for fluidized beds. The light alkane chromium-based dehydrogenation catalyst has a spinel structure and is used in a fluidized bed reactor with a reaction pressure of 0.01-0.50 MPa, a temperature of 530-660° C., and a volume space velocity of 800-2400 h⁻¹.

The light alkane chromium-based dehydrogenation catalyst has a spinel structure, including at least one of a magnesia-alumina spinel structure, a calcium aluminum spinel structure, or a titanium containing spinel structure.

The light alkane chromium-based dehydrogenation catalyst comprises the following components in mass fractions based on the total weight on a dry basis of the light alkane chromium-based dehydrogenation catalyst: 0.1%-35% of chromium oxide, 0.1%-5% of a first promoter, 0.1%-10% of a second promoter, 0.1%-5% of a third promoter, and the balance being a fluidized bed carrier;

Preferably, the following components in mass fractions based on the total weight on a dry basis: 0.1-35% of chromium oxide, 0.1-5% of a first promoter, 0.1-10% of a second promoter, 0.1-5% of a third promoter, and the balance being a fluidized bed carrier; and more preferably, 1-25% of chromium oxide, 0.5-3% of a first promoter, 0.5-3% of a second promoter, 0.5-3% of a third promoter, and the balance being a fluidized bed carrier.

Among them, chromium in chromium oxide is derived from one or more of sodium chromate, sodium dichromate, potassium chromate, potassium dichromate, ammonium dichromate, chromic acid, chromium chloride, acetylacetonate chromic acid, potassium chromium sulfate, chromium trioxide, chromium peroxide, lead chromate, chromium nitride, chromium nitrate and chromium fluoride.

The first promoter is a substance containing at least one alkaline earth metal element, and comprises one or more of alkaline earth metal powders, alkaline earth metal halides, alkaline earth metal oxides, alkaline earth metal sulfides, alkaline earth metal nitrates, alkaline earth metal acetates and alkaline earth metal oxalates. Preferably, the first promoter is alkali metal acetate and nitrate, when such substances are used as the first promoter, it is not easy to introduce impurities during the preparation of the catalyst.

The second promoter is a substance containing at least one Group IVB element, and comprises one or more of metal powders of Group IVB elements, halides of Group IVB elements, oxides of Group IVB elements, sulfides of Group IVB elements, nitrates of Group IVB elements, acetates of Group IVB elements and oxalates of Group IVB elements. Preferably, the second promoter is an organic ammonium salt and nitrate salt of the Group IVB elements, when such substances are used as the second promoter, it is not easy to introduce impurities during the preparation of the catalyst.

The third promoter is a substance containing at least one lanthanide metal element, and comprises one or more of lanthanide metal powders, lanthanide metal halides, lanthanide metal oxides, lanthanide metal sulfides, lanthanide metal carbides, lanthanide metal nitrates, lanthanide metal carbonates, and lanthanide metal oxalates. Preferably, the third promoter is lanthanide metal nitrates and carbonates, when such substances are used as the third promoter, it is not easy to introduce impurities during the preparation of the catalyst.

Further, the fluidized bed carrier comprises alumina, molecular sieve or silica with high mechanical strength. The specific surface area of the fluidized bed carrier is 50-300 m²/g, and the particle size is 100-200 μm. Preferably, the specific surface area of the fluidized bed carrier is 80-120 m²/g, and the particle size is 120-180 μm. Using this specification of fluidized bed carrier is helpful for fluidization of catalysts.

This embodiment also provides a preparation method of the light alkane chromium-based dehydrogenation catalyst, comprising:
(1) mixing chromium oxide, a first promoter, a second promoter, and a third promoter in mass fractions to obtain an impregnating solution; and
(2) under vacuum conditions, impregnating the fluidized bed carrier in the impregnation solution, then aging for 1-10 hours when the surface was dry, drying, and then calcining at 650-950° C. for 2-10 hours.

Further, the required soluble solution containing chromium can be formulated, and its chromium source can use one of oxides of chromium, acetates of chromium, nitrates of chromium, and oxalates of chromium; the required soluble solution containing calcium or magnesium can be formulated, and the magnesium source can use one of sulfates containing magnesium, chlorides containing magnesium and nitrates containing magnesium. When the required soluble solution containing calcium is formulated, the calcium source can use one of chlorides containing calcium, nitrates containing calcium and sulfates containing calcium.

Further, during the impregnation process of the fluidized bed carrier with the impregnating solution, the vacuum degree is preferably between 0.2 kPa-50 kPa, and more preferably between 0.2 kPa-5 kPa; the aging time is preferably 1-5 hours, and more preferably 2-4 hours; the drying time is preferably 2-8 hours, and more preferably 2-5 hours; the calcining temperature is preferably 750-950° C., and more preferably 750-900° C.; and the calcining time is preferably 2-8 hours, and more preferably 4-6 hours.

The specific embodiments of the present invention are described in detail below. It should be understood that the specific embodiments described herein are only used to illustrate and explain the present invention, and are not used to limit the present invention.

### Example 1

This example provides a light alkane chromium-based dehydrogenation catalyst for fluidized beds, the preparation method of which includes:
(1) Oven-drying the aluminite powder and raising the temperature to 900° C. at a rate of 5° C./min, calcining at this temperature for 3 hours, cooling, then sieving to obtain a fluidized bed carrier with a particle size of 100-200 μm, wherein the measured water absorption was 45 g H₂O/100 g and the specific surface area was 110-120 m²/g.
(2) Weighing 23.8 g of chromic anhydride, 6.4 g of magnesium acetate tetrahydrate, 2.2 g of ammonium titanyl oxalate monohydrate, and 3.2 g of lanthanum nitrate hexahydrate and dissolving them in 50 g of water to obtain the impregnation solution; adding 100 g of the fluidized bed carrier into a suction flask, vacuumizing for 2 hours at a vacuum degree of −50 kPa; slowly adding the impregnation solution into the suction flask, and stirring the wet fluidized bed carrier every 10 minutes until the surface of the carrier was air dried. Oven-drying the catalyst in an oven at 110° C. for 8 hours to obtain a dried catalyst; calcining the dried catalyst in a muffle furnace at a calcining temperature of 800° C. for a calcining time of 8 hours, with a heating rate of 5° C./min, and the fluidized bed catalyst 1 was obtained when the catalyst naturally cooled down to below 200° C.

### Example 2

This example provides a light alkane chromium-based dehydrogenation catalyst for fluidized beds, the preparation method of which includes:
weighing 33.9 g of chromic anhydride, 6.8 g of magnesium acetate tetrahydrate, 1.7 g of zirconium oxychloride octahydrate, and 3.4 g of lanthanum nitrate hexahydrate and dissolving them in 50 g of water to obtain the impregnation solution; adding 100 g of the fluidized bed carrier prepared in Example 1 into a suction flask, vacuumizing for 2 hours at a vacuum degree of −50 kPa; slowly adding the impregnation solution into the suction flask, and stirring the wet fluidized bed carrier every 10 minutes until the surface of the carrier was air dried. Oven-drying the catalyst in an oven at 110° C. for 8 hours to obtain a dried catalyst; calcining the dried catalyst in a muffle furnace at a calcining temperature of 650° C. for a calcining time of 6 hours, with a heating rate of 5° C./min, and the fluidized bed catalyst 2 was obtained when the catalyst naturally cooled down to below 200° C.

### Example 3

This example provides a light alkane chromium-based dehydrogenation catalyst for fluidized beds, the preparation method of which includes:
weighing 33.7 g of chromic anhydride, 5.4 g of calcium nitrate tetrahydrate, 1.7 g of zirconium oxychloride octahydrate, and 1.6 g of cerium nitrate hexahydrate and dissolving them in 50 g of water to obtain the impregnation solution; adding 100 g of the fluidized bed carrier prepared in Example 1 into a suction flask, vacuumizing for 2 hours at a vacuum degree of −50 kPa; slowly adding the impregnation solution into the suction flask, and stirring the wet fluidized bed carrier every 10 minutes until the surface of the carrier was air dried. Oven-drying the catalyst in an oven at 110° C. for 8 hours to obtain a dried catalyst; calcining the dried catalyst in a muffle furnace at a calcining temperature of 750° C. for a calcining time of 6 hours, with a heating rate of 5° C./min, and the fluidized bed catalyst 3 was obtained when the catalyst naturally cooled down to below 200° C.

### Example 4

This example provides a light alkane chromium-based dehydrogenation catalyst for fluidized beds, the preparation method of which includes:
weighing 29.8 g of chromic anhydride, 4 g of barium chloride dihydrate, 2.3 g of ammonium titanyl oxalate monohydrate, and 1.6 g of cerium nitrate hexahydrate and dissolving them in 50 g of water to obtain the impregnation solution; adding 100 g of the fluidized bed carrier prepared in Example 1 into a suction flask, vacuumizing for 2 hours at a vacuum degree of −50 kPa; slowly adding the impregnation solution into the suction flask, and stirring the wet fluidized bed carrier every 10 minutes until the surface of the carrier was air dried. Oven-drying the catalyst in an oven at 110° C. for 8 hours to obtain a dried catalyst; calcining the dried catalyst in a muffle furnace at a calcining temperature of 850° C. for a calcining time of 6 hours, with a heating rate of 5° C./min, and the fluidized bed catalyst 4 was obtained when the catalyst naturally cooled down to below 200° C.

### Example 5

This example provides a light alkane chromium-based dehydrogenation catalyst for fluidized beds, the preparation method of which includes:
(1) using ZSM-5 molecular sieve as a carrier and oven-drying the molecular sieve, then raising the temperature to 500° C. at a rate of 5° C./min, calcining at this temperature for 2 hours, cooling, then sieving to obtain a fluidized bed carrier with a particle size of 100-200 μm, wherein the measured water absorption was 45 g H₂O/100 g and the specific surface area was 50-60 m²/g.
(2) Weighing 23.8 g of chromic anhydride, 6.4 g of magnesium acetate tetrahydrate, 2.2 g of ammonium titanyl oxalate monohydrate, and 3.2 g of lanthanum nitrate hexahydrate and dissolving them in 50 g of water to obtain the impregnation solution; adding 100 g of the fluidized bed carrier into a suction flask, vacuumizing for 2 hours at a vacuum degree of −40 kPa; slowly adding the impregnation solution into the suction flask, and stirring the wet fluidized bed carrier every 5 minutes until the surface of the carrier was air dried. Oven-drying the catalyst in an oven at 120° C. for 2 hours to obtain a dried catalyst; calcining the dried catalyst in a muffle furnace at a calcining temperature of 950° C. for a calcining time of 2 hours, with a heating rate of 10° C./min, and the fluidized bed catalyst 5 was obtained when the catalyst naturally cooled down to below 200° C.

### Example 6

This example provides a light alkane chromium-based dehydrogenation catalyst for fluidized beds, the preparation method of which includes:
(1) Using porous silica as a carrier and oven-drying the silica, then raising the temperature to 300° C. at a rate of 5° C./min, calcining at this temperature for 10 hours, cooling, then sieving to obtain a fluidized bed carrier with a particle size of 100-200 μm, wherein the measured water absorption was 45 g H₂O/100 g and the specific surface area was 250-300 m²/g.
(2) Weighing 33.7 g of chromic anhydride, 5.4 g of calcium nitrate tetrahydrate, 1.7 g of zirconium oxychloride octahydrate, and 1.6 g of cerium nitrate hexahydrate and dissolving them in 50 g of water to obtain the impregnation solution; adding 100 g of the fluidized bed carrier into a suction flask, vacuumizing for 2 hours at a vacuum degree of −100 kPa; slowly adding the impregnation solution into the suction flask, and stirring the wet fluidized bed carrier every 20 minutes until the surface of the carrier was air dried. Oven-drying the catalyst in an oven at 100° C. for 5 hours to obtain a dried catalyst; calcining the dried catalyst in a muffle furnace at a calcining temperature of 650° C. for a calcining time of 10 hours, with a heating rate of 3° C./min, and the fluidized bed catalyst 6 was obtained when the catalyst naturally cooled down to below 200° C.

### Comparative Example 1

This comparative example provides a dehydrogenation catalyst, the preparation method of which includes:

Weighing 23.7 g of chromic anhydride, 6.4 g of magnesium acetate tetrahydrate, and dissolving them in 50 g of water to obtain the impregnation solution; adding 100 g of the fluidized bed carrier into a suction flask, vacuumizing for 2 hours at a vacuum degree of −50 kPa; slowly adding the impregnation solution into the suction flask, and stirring the wet fluidized bed carrier every 10 minutes until the surface of the carrier was air dried. Oven-drying the catalyst in an oven at 110° C. for 8 hours to obtain a dried catalyst; calcining the dried catalyst in a muffle furnace at a calcining temperature of 650° C. for a calcining time of 6 hours, with a heating rate of 5° C./min, and the comparative fluidized bed catalyst 1 was obtained when the catalyst naturally cooled down to below 200° C.

### Comparative Example 2

This comparative example provides a dehydrogenation catalyst, the preparation method of which includes:

Weighing 29.8 g of chromic anhydride, 6.7 g of magnesium acetate tetrahydrate, 2.3 g of titanyl oxalate monohydrate and dissolving them in 50 g of water to obtain the impregnation solution; adding 100 g of the fluidized bed carrier into a suction flask, vacuumizing for 2 hours at a vacuum degree of −50 kPa; slowly adding the impregnation solution into the suction flask, and stirring the wet fluidized bed carrier every 10 minutes until the surface of the carrier was air dried. Oven-drying the catalyst in an oven at 110° C. for 8 hours to obtain a dried catalyst; calcining the dried catalyst in a muffle furnace at a calcining temperature of 850° C. for a calcining time of 6 hours, with a heating rate of 5° C./min, and the comparative fluidized bed catalyst 2 was obtained when the catalyst naturally cooled down to below 200° C.

To demonstrate the technical effects of the light alkane chromium-based dehydrogenation catalyst provided in this application, the following experiments were conducted specially:

### Experimental Example 1 Propane Dehydrogenation Test

Propane dehydrogenation tests were conducted on the fluidized bed catalysts 1-4 prepared in Examples 1-4 and the comparative fluidized bed catalysts 1-2 in Comparative Examples 1 and 2, respectively;

The adopted process flow is the existing process flow, which will not be elaborated in detail in the examples. The control parameters in the process flow are as follows: propane volume space velocity was 1000 h⁻¹, an appropriate amount of nitrogen gas was introduced, the partial pressure of propane was maintained at 50 kPa, and the total pressure of the reaction system was atmospheric pressure; the bed temperature was 560-610° C.; and the results are shown in Table 1,

TABLE 1

| | | | | | Conversion | |
|---|---|---|---|---|---|---|
| | | | | | rate of | Propene |
| Serial | | A second | A third | | propane | selectivity |
| number | A first promoter | promoter | promoter | Carrier | wt % | wt % |
| Example 1 | Magnesium | Ammonium | Lanthanum | Alumina | 45.26 | 86.76 |
| | acetate | titanyl oxalate | nitrate | | | |
| | tetrahydrate | monohydrate | hexahydrate | | | |
| Example 2 | Magnesium | Zirconium | Lanthanum | Alumina | 42.26 | 85.27 |
| | acetate | oxychloride | nitrate | | | |
| | tetrahydrate | octahydrate | hexahydrate | | | |
| Example 3 | Calcium nitrate | Zirconium | Cerium nitrate | Alumina | 46.02 | 84.16 |
| | tetrahydrate | oxychloride | hexahydrate | | | |
| | | octahydrate | | | | |
| Example 4 | Barium chloride | Ammonium | Cerium nitrate | Alumina | 45.81 | 86.82 |
| | dihydrate | titanyl oxalate | hexahydrate | | | |
| | | monohydrate | | | | |
| Comparative | Magnesium | | / | Alumina | 44.73 | 76.88 |
| Example 1 | acetate | | | | | |
| | tetrahydrate | | | | | |
| Comparative | Magnesium | Ammonium | | Alumina | 40.93 | 83.05 |
| Example 2 | acetate | titanyl oxalate | | | | |
| | tetrahydrate | monohydrate | | | | |

It can be seen from Table 1 that compared to the catalysts provided in Comparative Examples 1 and 2, the light alkane chromium-based dehydrogenation catalysts provided in Examples 1-4 of the present invention have improved propylene selectivity in the propane dehydrogenation reaction at 600° C.

### Experimental Example 2

The process flow adopted in propane dehydrogenation performance test of fluidized bed catalyst 3 provided in Example 3 at different temperatures was the existing process flow, which will not be elaborated in detail in the examples. The control parameters in the process flow were as follows: propane volume space velocity was 1000 h⁻¹, an appropriate amount of nitrogen gas was introduced, the partial pressure of propane was maintained at 50 kPa, and the total pressure of the reaction system was atmospheric pressure; the bed temperature was 560-610° C.; and the results are shown in Table 2,

TABLE 2

| | | Conversion | | | |
|---|---|---|---|---|---|
| | | rate of | Propane | Propene | C1-C6 |
| Serial | | propane | selectivity | yield | byproduct |
| number | Temperature | wt % | wt % | wt % | wt % |
| Example 3 | 610 | 51.81 | 79.62 | 41.25 | 10.56 |
| | 600 | 48.35 | 83.16 | 40.21 | 8.14 |
| | 590 | 44.23 | 85.23 | 37.70 | 6.53 |
| | 580 | 41.94 | 87.66 | 36.76 | 5.18 |
| | 570 | 39.55 | 89.12 | 35.25 | 4.30 |
| | 560 | 34.97 | 92.36 | 32.30 | 2.67 |

As can be seen from Table 2 that such a light alkane chromium-based dehydrogenation catalyst exhibits an increase in conversion rate of catalyst and a decrease in selectivity as the temperature rises within the bed temperature range of 560 to 610° C., but the amount of by-products increases, which is consistent with the kinetic law of propane dehydrogenation catalysts. When the reaction temperature reaches 600° C., the yield of propylene is higher than 40%, exceeding that of traditional chromium-based dehydrogenation catalysts, thereby indicating that the chromium-based dehydrogenation catalyst prepared by the present invention has high reaction activity and stability.

Finally, it should be noted that the above is only preferred examples of the present invention and is not intended to limit the scope of protection of the present invention. Any modifications, equivalent substitutions, improvements, etc. made within the spirit and principles of the present invention should be included in the scope of protection of the present invention.

1. A light alkane chromium-based dehydrogenation catalyst, wherein the dehydrogenation catalyst has a spinel structure and comprises the following components in mass fractions based on the total weight on a dry basis:
0.1-35% of chromium oxide, 0.1-5% of a first promoter, 0.1-10% of a second promoter, 0.1-5% of a third promoter, and the balance being a carrier;
the first promoter is a substance containing at least one alkaline earth metal element;
the second promoter is a substance containing at least one Group IVB element; and
the third promoter is a substance containing at least one lanthanide metal element.

2. The light alkane chromium-based dehydrogenation catalyst according to claim 1, wherein the carrier has a specific surface area of 50-300 m²/g and a particle size of 100-200 μm.

3. The light alkane chromium-based dehydrogenation catalyst according to claim 2, wherein the carrier comprises at least one of alumina, molecular sieve and silica.

4. The light alkane chromium-based dehydrogenation catalyst according to claim 1, wherein the spinel structure comprises at least one of a magnesia-alumina spinel structure, a calcium aluminum spinel structure, a titanium containing spinel structure, and a perovskite structure.

5. The light alkane chromium-based dehydrogenation catalyst according to claim 1, wherein the first promoter comprises one or more of alkaline earth metal, alkaline earth metal halides, alkaline earth metal oxides, alkaline earth metal sulfides, alkaline earth metal nitrates, alkaline earth metal acetates and alkaline earth metal oxalates.

6. The light alkane chromium-based dehydrogenation catalyst according to claim 1, wherein the second promoter comprises one or more of metal of Group IVB elements, halides of Group IVB elements, oxides of Group IVB elements, sulfides of Group IVB elements, nitrates of Group IVB elements, acetates of Group IVB elements and oxalates of Group IVB elements.

7. The light alkane chromium-based dehydrogenation catalyst according to claim 1, wherein the third promoter comprises one or more of lanthanide metal, lanthanide metal halides, lanthanide metal oxides, lanthanide metal sulfides, lanthanide metal carbides, lanthanide metal nitrates, lanthanide metal acetates and lanthanide metal oxalates.

8. A preparation method of the light alkane chromium-based dehydrogenation catalyst according to claim 1, comprising:
mixing the chromium oxide, the first promoter, the second promoter, and the third promoter in mass fractions to obtain an impregnating solution; and
under vacuum conditions, impregnating the carrier in the impregnation solution, then aging for 1-10 hours, drying, and then calcining at 650-950° C. for 2-10 hours.

9. The preparation method of the light alkane chromium-based dehydrogenation catalyst according to claim 8, wherein the particle size of the carrier is 100-200 μm, which is obtained by calcining alumina, molecular sieve or silica at 300-1000° C. for 2-10 hours and sieving.

10. Application of the light alkane chromium-based dehydrogenation catalyst according to claim 1, wherein the catalyst is used in a fluidized bed reactor with a reaction pressure of 0.01-0.50 MPa, a temperature of 530-660° C., and a volume space velocity of 800-2400 h⁻¹.
